(19) United States
(12) Patent Application Publication
(10) Pub. No.: US 2025/0235529 A1
(43) Pub. Date: Jul. 24, 2025

(54) SARS-CoV-2 VACCINE COMPOSITIONS

(71) Applicant: MERCIA PHARMA, INC., New York, NY (US)

(72) Inventors: Peter BLACKBURN, New York, NY (US); Dimitrios DRIVAS, New York, NY (US)

(21) Appl. No.: 18/856,293

(22) PCT Filed: Apr. 11, 2023
(86) PCT No.: PCT/US23/65636

§ 371 Date: Oct. 11, 2024

Related U.S. Application Data
(60) Provisional application No. 63/380,499, filed on Oct. 21, 2022.
(60) Provisional application No. 63/329,851, filed on Apr. 11, 2022.
(60) Provisional application No. 63/329,850, filed on Apr. 11, 2022.

(51) Int. Cl. A61K 39/215; A61K 9/107; A61K 39/00; A61K 47/18; A61K 47/20; A61P 31/14; C12N 7/00

(52) U.S. Cl. CPC A61K 39/215; A61K 9/1075; A61K 47/18; A61K 47/20; A61P 31/14; C12N 7/00; A61K 2039/55566; A61K 2039/70; C12N 2770/20034

(57) ABSTRACT

The present disclosure provides compositions of adjuvanted SARS-CoV-2 vaccines and their use to prevent and manage Covid-19 infection, including host hyperinflammatory responses to infection, including long term symptoms associated with Covid infection.

### CROSS REFERENCE TO RELATED APPLICATIONS

This application claims priority benefit of U.S. Provisional Applications Ser. Nos. 63/329,850 filed Apr. 11, 2022, 63/329,851 filed Apr. 11, 2022 and 63/380,499 filed Oct. 21, 2022, the entire disclosures of each of which are incorporated herein by reference in their entireties.

### TECHNICAL FIELD

The field relates to compositions of adjuvanted SARS-CoV-2 vaccines and their use to prevent and manage Covid-19 infection, including host hyperinflammatory responses to infection, including long term symptoms associated with Covid infection.

### BACKGROUND

The Severe Acute Respiratory Syndrome coronavirus 2 (SARS-CoV-2) pandemic has caused the deaths of over five million people world-wide, and resulted in significant disruptions to the world economy. While the development of safe and effective vaccines has greatly contributed to the control of the pandemic, there is a continuing need for vaccines that confer improved protection at dose-sparing levels of antigen(s), that exhibit cross-reactivity towards emergent variants of concern (VOC), with superior durability of the overall immune response, particularly in vulnerable populations including immunocompromised and elderly subjects who remain particularly at-risk for hospitalization and death form Covid-19 infection. One potential solution to address these problems is the use of adjuvants in combination with various proteinaceous antigenic preparations related to the virus, including for example, the Acetyl choline (ACE-2) Receptor Binding Domain (RBD) of the so-called virus Spike protein or the full length stabilized trimeric Spike protein (S-protein).

Several different adjuvants are currently used in U.S. FDA approved vaccines. Aluminum based adjuvants, in one or more of amorphous aluminum hydroxyphosphate sulfate (AAHS), aluminum hydroxide, aluminum phosphate, and potassium aluminum sulfate (Alum), are used in a variety of vaccines, including Anthrax, DT, DTaP (Daptacel). DTaP (Infanrix). DTaP-IPV (Kinrix). DTaP-IPV (Quadracel). DTaP-HcpB-IPV (Pediarix), DTaP-IPV/Hib (Pentacel). Hep A (Havrix), Hep A (Vaqta). Hep B (Engerix-B). Hep B (Recombivax). HepA/Hep B (Twinrix). HIB (PedvaxHIB). HPV (Gardasil 9), Japanese encephalitis (Ixiaro), MenB (Bexsero, Trumenba), Pneumococcal (Prevnar 13), Td (Tenivac), Td (Mass Biologics). Tdap (Adacel), and Tdap (Boostrix).

MF59, an oil-in-water emulsion adjuvant containing squalene, is included in Fluad, a trivalent and/or quadrivalent inactivated vaccine against seasonal influenza licensed for use in adults older than 65 years of age. AS01 is a liposome-based adjuvant used in the shingles vaccine Shingrix, which contains two immunostimulants, 3-O-desacyl-4′-monophosphoryl lipid A (MPL), a non-toxic derivative of the lipopolysaccharide from Salmonella minnesota) and QS-21 (a saponin fraction extracted from Quillaja saponaria Molina). AS03 is an adjuvant system composed of α-tocopherol, squalene and polysorbate 80 in an oil-in-water emulsion, and is used in influenza vaccines Pandemrix and Arepanrix for use with the 2009 emergent A/H1N1 pandemic influenza strain. Cytosine phosphoguanosine (CpG) 1018, a toll-like receptor 9 (TLR9) agonist, is used in Heplisav-B vaccine. It is made up of cytosine phosphoguanine (CpG) motifs, which is a synthetic form of DNA that mimics bacterial and viral genetic material, and stimulates pro-inflammatory cytokines and a Th1 driven immune response.

Adjuvants are typically employed to promote enhanced antibody and cellular immune responses towards antigens which by themselves are insufficiently immunogenic. Adjuvants variously induce biased Th1 or Th2 type immune responses. Limitations however exist with respect to these currently available vaccine adjuvants, particularly with respect to the promotion of durable and cross reactive immunity that is expressed in at-risk populations, especially immunocompromized populations, including elderly subjects; limitations similarly exhibited by currently approved Covid vaccines based on mRNA and adenovirus vectored DNA vaccine technologies. However, despite their previous widespread use, the precise molecular mechanisms by which the available adjuvants actually work in humans is not well understood, and varies from antigen to antigen between various adjuvants requiring an empirical trial and error approach to formulation choice and vaccine development strategy be undertaken.

### SUMMARY

The present disclosure provides an adjuvanted SARS-CoV-2 vaccine. A SARS-CoV-2 proteinaceous antigen is formulated as a water-in-oil nanoglobular emulsion vaccine composition comprising an aqueous phase and an oil phase. The buffered aqueous phase comprises 25% to 35% by weight of the emulsion in the form of aqueous nanoglobules having a median diameter from about 0.3 μm to about 1 μm, comprising one or more SARS-CoV-2 antigens. The oil phase comprises from 65% to 75% by weight of the emulsion, and comprises 85% to 90% of squalene and squalane, 9% to 12% mannide monooleate, and 0.5% to 0.7% polyoxyl-40-hydrogenated castor oil, each by weight of the oil phase.

In some embodiments, the SARS-CoV-2 antigen is a protein antigen. In some embodiments, the nanoemulsion vaccine composition comprises more than one SARS-CoV-2 antigen. In some embodiments, the SARS-CoV2 antigen comprises components derived from more than one SARS-CoV-2 strain. In some embodiments, the SARS-CoV2 antigen comprises SARS-CoV-2 antigens derived from more than one SARS-CoV-2 strain.

In some embodiments, squalene comprises a range from about 40% to about 60% by weight of the oil phase. In some embodiments, squalane comprises a range from about 40% to about 60% by weight of the oil phase. In some embodiments, the squalene and squalane are in a ratio of about 1:1 by weight.

In some embodiments, the median globule size is about 300 nm. In some embodiments, the composition has a viscosity of 100 cP or less. In some embodiments, the aqueous phase further comprises a protein solubilizer. In some embodiments, the protein solubilizer is urea or DMSO.

The present disclosure further provides a method for SARS-CoV-2 prophylaxis comprising administering to a patient in need thereof a vaccine composition as described herein. The present disclosure further provides a method for attenuating SARS-CoV-2 infection comprising administering to a patient in need thereof a vaccine composition as described herein. The present disclosure further provides a method for producing an immune response protective against SARS-CoV-2 infection comprising administering to a patient in need thereof a vaccine composition as described herein.

The present disclosure further provides a method for long-haul SARS-CoV-2 prophylaxis comprising administering to a patient in need thereof a vaccine composition as described herein. The present disclosure further provides an immunotherapeutic method for attenuating one or more symptoms of long-haul SARS-CoV-2 infection comprising administering to a patient in need thereof a therapeutic vaccine composition as described herein. The present disclosure further provides a method for producing an immune response protective against long-haul SARS-CoV-2 infection comprising administering to a patient in need thereof a vaccine composition as described herein. The present disclosure further provides a method for producing an immune response protective against long-haul SARS-CoV-2 infection comprising administering to a patient in need thereof a vaccine composition as described herein that simultaneously stimulates both a durable antibody response towards viral fragments and a balanced anti-inflammatory response towards patients' long-haul hyperinflammation. The present disclosure further provides a method for increasing the potency and/or durability of an immune response to a SARS-CoV-2 antigen, comprising administering the antigen in a water-in-oil nanoemulsion vaccine composition as described herein.

The present disclosure further provides a kit for the point-of-use administration of a water-in-oil nanoemulsion vaccine against a SARS-CoV-2 infectious agent comprising: a vial of an adjuvant oil comprising mannide monooleate, squalene and squalane, a vial of aqueous PBS comprising SARS-CoV-2 antigen, at least one syringe, a lipid resistant three-way stopcock, at least one needle, and a vial for storing the formulated water-in-oil nanoemulsion vaccine. In some embodiments, the kit is for the point-of-use administration of a nanoparticulate water-in-oil emulsion vaccine against said SARS-CoV-2 infectious agent comprising: a vial of an adjuvant oil comprising mannide monooleate, squalene and squalane, a vial of aqueous PBS for combining with an antigen, two syringes, a lipid resistant three-way stopcock, two needles and a vial for storing the formulated water-in-oil emulsion vaccine. In some embodiments, the nanoparticulate water-in-oil emulsion vaccine produced by the combination of the kit components is a composition as described herein. The present disclosure further provides a water-in-oil nanoemulsion vaccine composition produced by the components of the kits described above.

The vaccine emulsion may be prepared in bulk by batch homogenization or pulsed continuous flow-through homogenization procedures, or in single or repeat dose vials at point-of-use (POU) by a hand-held procedure. The POU preparation of the vaccine emulsion enables sparing antigen and oil phase supplies and facilitates vaccine formulation adjustments to be made as a pandemic evolves as a result of viral mutations that produce emergent VOCs that may evade existing vaccine-conferred or prior infection-derived immunity

The vaccine may be administered as a single or as multiple primary doses in immunologically naïve individuals. Alternatively, the vaccine may be administered as a booster dose to individuals previously vaccinated with heterologous vaccines such as mRNA-based or DNA-based vaccines. Furthermore, the vaccine may be administered to individuals exhibiting long-term symptoms (Long Haul Covid) in an immunotherapeutic modality whereby hyperinflammatory symptoms are suppressed towards a balanced Th1/Th1 humoral and cellular immune profile.

### BRIEF DESCRIPTION OF THE DRAWINGS

FIG. 1.A describes the Mean Minimum Log End-point Titer through Day 168 post-vaccination (d0, d28) for Groups 3 (10 μg CoV-2 RBD adsorbed on alum), Group 7 (10 μg CoV-2 RBD in MAS-1), and Group 9 (10 μg CoV-1 RBD in MAS-1).

FIG. 2.A describes Individual Minimum Log End-point Titer through day 168 post-vaccination (d0, d28), Group 3 CoV-2 10 μg RBD alum-adsorbed Positive Control

FIG. 2.B describes Individual Minimum Log End-point Titer through day 168 post-vaccination (d0, d28), Group 7 CoV-2 10 μg RBD in MAS-1.

FIG. 2.C describes Individual Minimum Log End-point Titer through day 168 post-vaccination (d0, d28), Group 9 CoV-1 10 μg RBD in MAS-1.

FIG. 3.A describes Group 3 Alum-adsorbed CoV-2 RBD: Comparison of Mean Minimum Log Titers towards CoV-2 RBD with Cross-reactive Titers towards CoV-1 RBD

FIG. 3.B describes Group 7 MAS-1 adjuvanted CoV-2 RBD: Comparison of Mean Minimum Log Titers towards CoV-2 RBD with Cross-reactive Titers towards CoV-1 RBD

FIG. 3.C describes Group 9 MAS-1 adjuvanted CoV-1 RBD: Comparison of Mean Minimum Log Titers towards CoV-1 RBD with Cross-reactive Titers towards CoV-2 RBD.

FIG. 4.A describes In Vitro Virus Neutralization Assay of Wild-type (WT-DG614G) CoV-2 Strain.

FIG. 4.B describes In Vitro Virus Neutralization Assay of Beta VOC (B.1.351) CoV-2 Strain.

FIG. 5.A describes Group 3 Alum adsorbed CoV-2 RBD: Comparison of Mean Log End-point Titers and IgG isotypes IgG1, IgG2a, IgG2b, and IgG 3 towards CoV-2 RBD.

FIG. 5.B describes Group 7 MAS-1 adjuvanted CoV-2 RBD: Comparison of Mean Log End-point Titers and IgG isotypes IgG1, IgG2a. IgG2b, and IgG 3 towards CoV-2 RBD.

FIG. 5.C describes Group 9 MAS-1 adjuvanted CoV-1 RBD: Comparison of Mean Log End-point Titers and IgG isotypes IgG1, IgG2a, IgG2b, and IgG 3 towards CoV-2 RBD.

### DETAILED DESCRIPTION

The vaccine composition of the present disclosure is comprised of a water-in-oil nanoemulsion wherein the vaccine antigen is contained within the buffered aqueous phase of the emulsion. The term “water-in-oil nanoemulsion” means an emulsion wherein the aqueous phase is dispersed in a continuous oil phase in the form of aqueous nanoglobules having a median diameter from about 0.3 μm to about 1 μm.

The term “RBD” means the SARS-CoV-2 spike protein Receptor Binding Domain that binds to the ACE-2 receptor on human and other mammalian cell surfaces to facilitate viral entry into the mammalian cell following membrane fusion.

The term Spike protein refers to the intact Covid virus protein that carries the RBD.

The vaccines of the invention can be produced either “point-of use” or as a “bulk filled” final drug product.

The vaccine emulsion may be prepared aseptically in bulk using sterile filtered components by repeat batch homogenization or by pulsed continuous flow-through homogenization procedures followed by sterile filling into single or repeat use vials that may be stored for up to three years at refrigerated (2-8° C.) temperatures. As a “point-of-use” product, the vaccine is formulated at room temperature as a nanoparticulate emulsion made by a simple, but robust and reproducible hand mixing procedure. This is illustrated by the globule size diameter 50% distribution results (D(v,0.5) for emulsions determined by laser light diffraction between multiple operators and their stability at room tem-perature. At T zero, globule size distribution for “point-of-use” emulsions prepared by 4 operators at the lower and upper mixing limits of the method were N=29; Mean D(v,0.5)±SEM=0.44±0.03 μm and N=26; Mean D(v,0.5)±SEM=0, 66±0.03 μm, respectively. Globule size measurements at 2, 18-24 and 48 hours after preparation indicated that there was no significant change in this parameter over the two-day storage period. To maintain sterile integrity the vaccine emulsion may be held at room temperature for up to 4 hours prior to injection permitting repeat dose volumes of up to 0.3 mL to be administered to multiple subjects per POU vial of vaccine emulsion

The components of the oil adjuvant vehicle suitable for use in the invention, comprise a first sugar ester emulsifier such as mannide monooleate (MMO) or sorbitan monooleate, a second emulsifier such as a hydrogenated castor oil, for example, polyoxyl-40-hydrogenated castor oil (POCO), and naturally occurring and metabolizable oils, preferably squalene and squalane. The metabolizable oils typically comprise from about 85% to about 90% by weight of the oil, the first sugar ester emulsifier from about 6% to 15%. i.e., about 9% to about 12%, or about 10% or 11% by weight of the oil, and the second emulsifier from about 0.1%-1.1%. i.e., 0.2% to about 1%, 0.4 to about 0.8%, 0.5% to about 0.7%, or about 0.6% by weight of the oil. The metabolizable oil component may be about 10%, to about 90% squalene, and about 10% to about 90% squalane by weight. In one embodiment the squalene to squalene are in a 1:1 ratio by weight.

The components of the oil vehicle, including their starting materials, which may be derived from cither animal or vegetable sources, or combinations thereof, are all commercially available from multiple sources. Preferably, components of the oil vehicle, including their starting materials, are derived from vegetable sources to avoid the risk of transmissible spongiform encephalitis (TSE) contamination. MAS-1 can be obtained from Mercia Pharma. Inc. Scarsdale. N.Y. (www.merciapharma.com).

Suitable sugar esters as the first emulsifier in addition to MMO include polysorbates, particularly sorbitan monooleate. In addition to POCO as the second emulsifier sorbitan esters, such as sorbitan monopalmitate, polysorbates, such as the Tweens family of emulsifiers, and Hypermers B239 and B246 may be useful.

The nanoparticulate vaccine emulsions of the invention typically contain from about 65% by weight to about 75% by weight of the adjuvant oil vehicle and about 25% to about 35% by weight of an aqueous phase containing the proteinaceous antigen. In certain embodiments of the invention the aqueous phase comprises from about 27% to about 33% by weight of the vaccine emulsion.

The water-in-oil vaccine emulsions used in the invention should be formulated so that the aqueous globules in the emulsion carrying the antigen have median diameters less than 1 micron with median diameters in the range from about 100 nanometers to about 1 micron, and typically with an average diameter of about 300 nanometers. The oil components of the adjuvant are preferably naturally occurring biological oils that are metabolizable, unlike the mineral oil that comprises the oil phase of the well known Freund's adjuvants (both incomplete and complete formulations).

The vaccine emulsions of the invention should tolerate high concentrations of antigen, such as from 0.1 mg/mL to 20 mg/mL, and should be compatible with commonly used protein solubilizers (e.g., 4M urea, 30% DMSO). Unlike IFA emulsions, they should be compatible with aqueous phases having a wide range of pH, i.e., from about 4-9, preferably 6-8, and should be unaffected over a wide range salt concentrations. Unlike high viscosity IFA emulsions (>1,500 cP), the vaccine emulsions of the invention should have a low viscosity (<100 cP) providing free flowing emulsions to permit high precision low volume (0.05 mL) dosing.

In some preferred embodiments, the vaccines of the present disclosure employ the Mercia Pharma MAS-1 adjuvant/delivery system. MAS-1 adjuvant sterile-filtered oil vehicle has a shelf life of at least 5 years stored at room temperature, making it suitable for stockpiling for pandemic preparedness, and facilitating its distribution without significant cold-chain concerns. Bulk manufactured MAS-1 adjuvanted vaccine emulsions have a shelf life of up to 3 years stored refrigerated at 2-8° C. MAS-1 adjuvant oil vehicle may also be formulated with aqueous solution containing antigen(s) by a rapid (90-120 seconds), robust and reproducible, validated point-of-use (POU) manual method.

MAS-1 adjuvanted emulsions are comprised of antigen-containing aqueous globules (300 nm diameter) dispersed in the continuous oil phase. The emulsions are free-flowing (viscosity<100 cP) allowing accurate dispensing of low volume doses of 0.05 to 0.5 mL, preferably 0.05 to 0.3 mL.

Thus, in certain embodiments, the water-in-oil nanoemulsion vaccine compositions of the present disclosure comprise an aqueous phase and an oil phase, wherein:
the aqueous phase comprises:
   25% to 35% by weight of the emulsion, and is in the form of aqueous nanoglobules having a median diameter from about 0.3 μm to about 1 μm; and
   one or more SARS-CoV-2 antigens; and
the oil phase comprises:
   from 65% to 75% by weight of the emulsion, which oil phase comprises 85% to 90% of squalene and squalane, 9% to 12% mannide monooleate, and 0.5% to 0.7% polyoxyl-40-hydrogenated castor oil, each by weight of the oil phase;
for example a MAS-1 adjuvanted vaccine.

In certain embodiments, the water-in-oil nanoemulsion vaccine compositions of the present disclosure comprise an oil phase, which comprises:
from 65% to 75% by weight of the emulsion, which oil phase comprises 85% to 90% of squalene and squalane, 9% to 12% mannide monooleate, and 0.5% to 0.7% polyoxyl-40-hydrogenated castor oil, each by weight of the oil phase;
for example a MAS-1 adjuvant oil vehicle.

The invention is further described in the following examples. The examples are merely illustrative and do not in any way limit the scope of the invention as described and claimed.

### EXAMPLES

### Overall Objectives

Evaluation the Potential of MAS-1 Adjuvanted Covid Vaccine to Stimulate Robust, Cross-Reactive. Durable and Balanced Th1/Th2-type Immune Responses towards SARS CoV-2 virus

### Limitations of Current Covid-19 Vaccines:

SARS CoV-2 is responsible for Covid-19 infections first arising in late 2019 in Wuhan, China, while SARS CoV-1 was responsible for the original SARS infections arising in South East Asia in 2003. Whereas SARS CoV-1 caused serious illness in a high percentage of infected individuals, and death in as many as 10 percent of infected individuals, CoV-1 was not highly contagious. However, although SARS CoV-2 is a less lethal virus than SARS CoV-1, it is a highly contagious virus and has been responsible for serious illness and death in many infected individuals on a world-wide basis. SARS CoV-2 is especially problematic in immunocompromised and elderly populations. Moreover, SARS CoV-2 is highly mutagenic and in the short time since it was first identified a number of variants have and continue to emerge. Each mutant has the potential to be more or less contagious, more or less pathogenic, and to varying degrees, to evade the host's immune system in individuals primed by prior natural exposure to CoV-2, or after immunization with a CoV-2 vaccine derived from existing strain(s). Moreover, the durability of the immune response following either natural exposure to coronaviruses in general, including CoV-2, or following vaccination with currently available CoV-2 vaccines, is limited and requires repeat boosting with mRNA vaccines after 4 to 6 months, and after adenovirus vaccines, as frequently as 2 months after the primary dose(s). Moreover, infection with SARS CoV-2 that leads to serious Covid-19 infections has been correlated with hyperimmune Th1 type inflammatory reactions characterized by a so-called inflammatory “cytokine storm” elicited by the host that leads to serious illness and death of the infected individual. Thus, a vaccination strategy that promotes a more balanced Th1/Th2 immune response is more likely to prime the host immune system to provide a more regulated protective immune response that reduces the likelihood for life-threatening host Th1-type hyperimmune reactions. A further complication associated with SARS CoV-2 infection is that a large percentage of individuals, including individuals who manifest mild to moderate symptoms initially following infection, proceed to develop a complexity and diverse array of long term symptoms, so called “Long Haul” Covid. The underlying causes of Long Haul symptoms is not yet well understood, but to the extent that the syndrome is elicited or propagated by the host's immune response to Covid-19 infection, or residual viral particulates and/or antigen, immunological intervention with a prophylactic or therapeutic vaccine that stimulates a more balanced Th1/Th2 immune response may have a beneficial role by regulating the hyperinflammatory Th1 response towards a more balanced Th1/Th2 state. Moreover, a prophylactic or therapeutic vaccine that stimulates a durable humoral response has the further potential to eliminate residual viral fragments that may also contribute to prolonged stimulation of the host inflammatory response.

Thus, the development of new and improved Covid-19 vaccines should ideally better address the challenges presented by the current Covid-19 virus strains and be responsive to the emergence of new variants by exhibiting a broader degree of cross-reactive protection, a response that is more durable and thereby requiring less frequent need for booster doses, and a response that is effective in high-risk populations such as immunocompromised individuals, such as individuals with cancer, and in elderly populations whose immune resilience is compromised by immunosenescence. Furthermore, an improved Covid-19 vaccine should stimulate a balanced Th1/Th2 type immune response to offset the potential for breakthrough infections to promote the hyperinflammatory “cytokine storm” associated with serious Covid-19 infections and Covid-19 deaths. Moreover, a vaccine that stimulates a balanced Th1/Th2 type immune response at humoral and cellular levels has the potential to rebalance host inflammatory profiles, and thereby potentially play a role in preventing the initiation and/or propogation and/or therapeutic reversal of “Long Haul” Covid symptoms.

### Overall Strategy

To address these concerns, in mouse models, we evaluated the ability of MAS-1 adjuvant to enhance the immunity towards both SARS Cov-1 RBD and SARS CoV-2 RBD antigens through a minimum of 6 months post-vaccination to assess both the potency and durability of the immune response.

To assess the potential for improved cross-reactivity towards future variants, we assessed the ability of anti-sera raised by MAS-1/CoV-2 RBD to cross-react with SARS CoV-1 RBD, and vice versa, the ability of ani-sera raised by MAS-1/CoV-1 RBD to cross-react with SARS CoV-2 RBD. The amino acid sequence of SARS CoV-1 differs from that of SARS CoV-2 in as much as 30% of its sequence, significantly more so than differences between currently emerged SARS CoV-2 variants of concern (VOC), including, Alpha. Beta. Delta, and Omicron, and thus represents an extreme challenge model to assess the potential of MAS-1 to induce cross-reactive protection towards Covid-19 mutants yet to emerge.

To assess the vaccine's potential to prime a balanced Th1/Th2 immune response to prevent the likelihood for hyperinflammatory “cytokine storm”, and/or to prevent the host inflammatory responses that potentially lead to/or propogate “Long Haul” Covid, symptoms, the IgG isotype response and cytokine profiles stimulated by MAS-1 adjuvanted Covid vaccine were evaluated.

Study Outline: A total of 9 groups of 9 female BALB/cJ mice were immunized with the test articles on day 0 and 28 as set forth in Table 1.. Each test article was administered i.m. in 2×0.05 mL doses into each rear thigh muscle. Sera were collected on days 0 (pre-dose 1) 14, 28 (pre-dose 2), 42, 56, 84 and 169. Two animals per group were euthanized by cardiac puncture on each of days 42 and 84 to harvest spleens for preparation of splenocytes. Sera from these cardiac bleeds were used to provide in-assay serum controls for the ELISA assays across all plates. The remaining 5 animals per group were euthanized on day 169 by cardiac puncture to collect 6 month sera to assess durability, harvest spleens to assess cellular immune responses, and to collect various tissues preserved in formalin and frozen for future toxicological evaluations to assess product safety. In addition, throughout the duration of the study animals were monitored for general well-being and body weights were measured weekly in order to assess the overall safety profile of the MAS-1 adjuvanted Covid vaccine formulations.

The following Test Groups were employed:

| | | |
|---|---|---|
| Group 1: | Tris Buffered Saline (TBS) | |
| | negative control | |
| Group 2: | MAS-1/TBS placebo control | |
| Group 3: | CoV-2 RBD adsorbed to alum | 10 μg RBD/0.1 mL dose |
| | positive control | |
| Group 4: | CoV-2 RBD on alum in MAS-1 | 5 μg RBD/0.1 mL dose |
| Group 5: | CoV-2 RBD on alum in MAS-1 | 1.5 μg RBD/0.1 ml dose |
| Group 6: | Cov-2 RBD on alum in MAS-1 | 0.5 μg RBD/0.1 mL dose |
| Group 7: | CoV-2 RBD in MAS-1 | 10 μg RBD/0.1 mL dose |
| Group 8: | CoV-1 RBD on alum in MAS-1 | 5 μg RBD/0.1 mL dose |
| Group 9: | CoV-1 RBD in MAS-1 | 10 μg RBD/0.1 mL dose |

For logistical reasons, the 9 groups were enrolled in three cohorts of three groups to facilitate the evaluations: Cohort 1: Groups 2, 3, 4; Cohort 2: Groups 5, 6, 7; Cohort 3: Groups 1, 8, 9.

### ELISA Assays

Preliminary estimates of titers were made on day 42 serially diluted sera from euthanized mice to establish the optimal initial dilutions to permit antibody titers to be evaluated for the various test sera. For groups 3, 4, and 8 the initial “optimal” dilution was determined to be 20-fold. For groups 7 and 9 the initial “optimal” dilution was determined to be 5,000-fold. End-point titer estimates were made on both a per animal basis and across all 9 animals.

In each case, the baseline “Threshold” was based on the Mean baseline OD450+3×the SD (“baseline” in this case is the OD450 values obtained from serum samples of either non-immunized or MAS-1 placebo-immunized mice assayed in the same ELISA assay format as test samples). The “maximum” log titer is that dilution which first exceeds the baseline “Threshold”; the “medium” log titer is that dilution 2 times lower than that which first exceeds the baseline “Threshold”. The “minimum” log end-point titer as reported herein was estimated as that serum dilution 3 times lower than that dilution which first exceeds the baseline “Threshold” OD450 value in order to conservatively estimate the minimum titer elicited by each test article.

### Pseudovirus Assay for the Determination of Neutralizing Antibodies

The day before the assay, HEK-293T-hACE2 cells were plated at 1.5×10⁴ cells in 100 μL DMEM media. Pseudoviruses were incubated with serial dilutions of the serum samples for 1 hour at 37° C. Mouse sera samples were first diluted 50-fold, followed by 3-fold serial dilutions for a total of seven dilutions. Rat sera samples were diluted first 25-fold, followed by 3-fold serial dilutions for a total of seven dilutions.

100 μL of sera-pseudovirus were added to 293T-hACE2 cells in 96-well poly-D-lysine coated culture plates. Following 48 hours of incubation in a 5% CO₂ environment at 37° C., the cells were lysed with 100 μL of Promega Glo Lysis buffer for 15 minutes at RT. Finally, 50 μL of the lysate were added to 50 μL luciferase substrate (Promega Luciferase Assay System). The amount of luciferase was quantified by luminescence (relative luminescence units (RLU)), using a Luminometer (Biosynergy H4).

The conditions were tested in duplicate wells on each plate, and a virus control (VC=no sera) and cell control (CC=no pseudovirus) were included on each plate to determine the value for 0% and 100% neutralization, respectively. The percentage of inhibition of infection for each dilution of the sample was calculated according to the RLU values as follows: % inhibition=[1−(average RLU of sample−average RLU of CC)/(average RLU of VC−average RLU of CC)]×100%. The 50% inhibitory dilution (EC50) was further defined as the serum dilution at which the relative light units (RLUs) were reduced by 50% compared with the virus control wells (virus+cells) after subtraction of the background RLUs in the control groups with cells only. The EC50 of each sample was calculated by the Reed-Muench method. (https://www.nature.com/articles/s41596-020-0394-5 (4)).

Human convalescent plasma was included as a positive control.

### Study Results

### Example 1: Potency and Durability of MAS-1-Induced Mean Log Minimum End-Point Titers

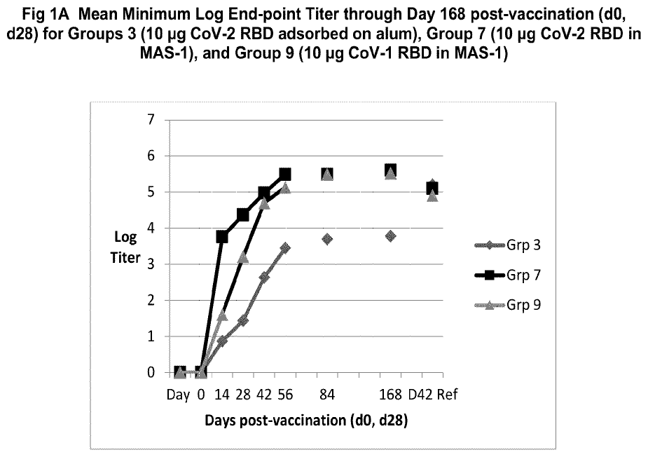

The mean log minimum end-point titers through day 168 post-vaccination (d0, d28) for Group 3 (10 μg CoV-2 RBD adsorbed on alum; positive control), Group 7 (10 μg CoV-2 RBD in MAS-1), and Group 9 (10 μg CoV-1 RBD in MAS-1.) are show in FIG. 1A. The mean minimum log end-point titer for Group 7, 10 μg CoV-2 RBD in MAS-1, compared with those for the positive control Group 3, 10 μg CoV-2 RBD adsorbed on alum, were at least 2 logs higher across all time points through day 168 (FIG. 1A). The mean minimum log end point titers for Group 9, 10 μg CoV-1 RBD in MAS-1 were also approximately 2 logs higher than those for Group 3 positive control by days 42, and also remained as such at all time points post-vaccination through day 168 with no evidence for diminution of titers (FIG. 1A).

### Example 2: Robustness of Mean Log Minimum End-Point Titers

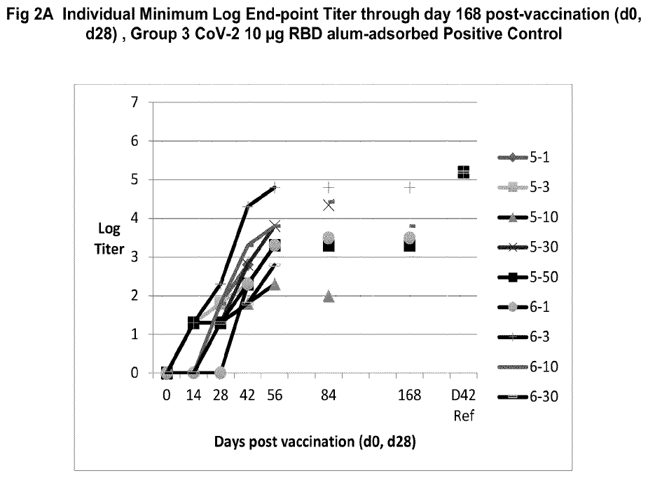

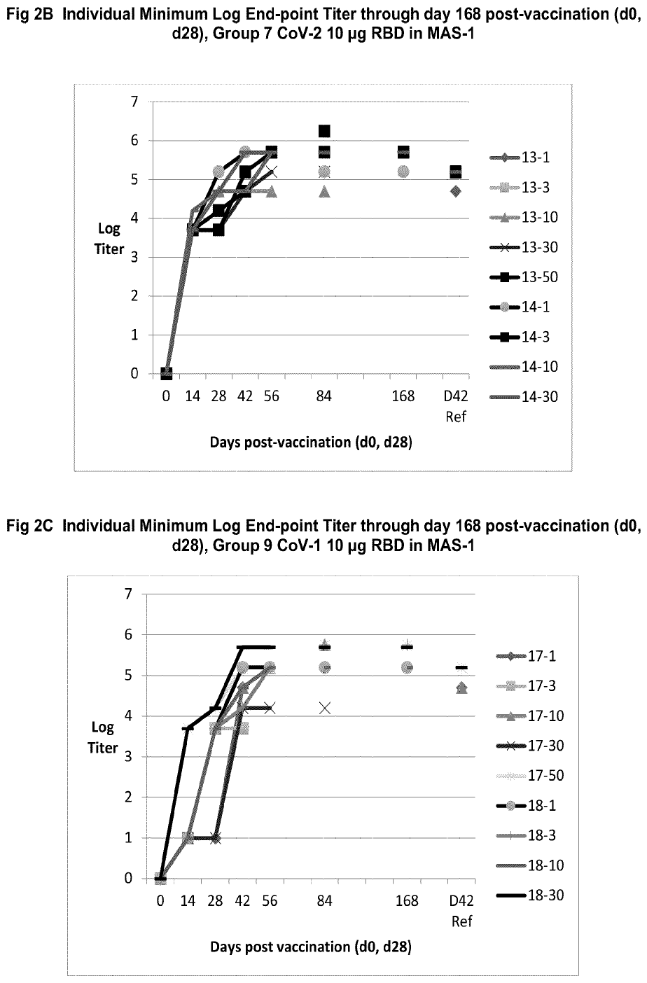

In order to assess the robustness of the immune response induced by the MAS-1-adjuvanted Covid vaccines, the log minimum end-point titers for individual animals in each of the groups are presented for each group in FIG. 2A, Group 3: 10 μg CoV-2 RBD adsorbed on alum positive control; FIG. 2B, Group 7: 10 μg CoV-2 RBD in MAS-1; FIG. 2C. Group 9: 10 μg CoV-1 RBD in MAS-1.

To maintain inter-assay robustness, positive control reference sera derived from group 7 and group 9 day 42 euthanized mice were run on each ELISA plate, respectively, as presented in FIG. 1A, and FIGS. 2A, 2B, and 2C. Likewise, MAS-1 placebo day 42 sera were run on each ELISA plate as negative controls in these studies. In addition, for each individual animal, day 42 sera were re-assayed as controls along with day 84 sera and day 84 titers were compared and normalized against the repeat day 42 titers. Likewise, for each animal, day 84 sera were re-assayed as controls along with day 168 sera and day 168 titers were compared and normalized against the repeat day 84 titers. There was high degree of conformity in the respective minimum log end-point titers assigned for these positive and baseline threshold values assigned to these negative controls across all ELISA plates and between different days the assays were performed throughout the study to day 168; confirming the robustness of the study design itself

Group 3 alum adsorbed positive control animals (FIG. 2A) exhibited a high degree of variability between individual animals, and in general, positive titers were observed only after the second dose administered on day 28, suggesting a lack of robustness in the immune response to CoV-2 RBD alum adsorbed positive control. Whereas, the minimum log end-point titers for Group 7 CoV-2 RBD in MAS-1 in all 9 individual animals (FIG. 2B) were robust even after only a single dose of CoV-2 RBD in MAS-1 achieving at least 4 log minimum titers by day 14 and day 28 pre-dose 2, suggesting the potential for a robust immunization regimen with just a single dose of CoV-2 RBD in MAS-1 may provide effective protection towards Covid-19. Similarly, although to a lesser extent than for Group 7, the minimum log end-point titers for Group 9 CoV-1 RBD in MAS-1 also exhibited a robust response with all animals expressing titers after a single dose of CoV-1 RBD in MAS-1 by day 14 and pre-dose 2 day 28 (FIG. 2C). Group 7 and Group 9 animals exhibited a further increase in minimum log end-point titers by day 42 following the second dose of MAS-1 adjuvanted CoV-2 and CoV-1 RBDs that was maintained without evidence for diminution though day 168, whereas, the variability between individual animals in Group 3 remained evident through day 168 towards alum-adsorbed CoV-2 RBD.

The data for Groups 7 and 9 further suggest that even a single dose regimen for MAS-1 adjuvanted CoV-2 RBD (and CoV-1 RBD) is likely to be effective in a clinical setting even as a primary dose. Moreover, these data suggests that MAS-1 adjuvanted CoV-2 or CoV-1 RBDs should be highly effective as a single dose for annual re-vaccination in a Covid-19 endemic setting as is currently performed with seasonal influenza vaccines.

Minimum log titers through day 84 for Group 4 animals immunized with alum adsorbed CoV-2 formulated in MAS-1 at 5 μg RBD dose, although comparable, were slightly lower than those for Group 3 alum adsorbed CoV-2 RBD at 10 μg dose. The minimum log end point titers for Group 8, alum adsorbed CoV-1 at 5 μg RBD dose, although comparable, were only slightly higher than those for Group 3 10 μg alum adsorbed CoV-2 RBD positive control (data not shown). As a result of the relatively poor response compared with Groups 7 and 9, the sera from Groups 4 and 8, as well as those from Groups 5 and 6 were not analyzed further.

Reproducibility between analyses across the time points was evaluated by use of common positive and negative controls included in each microtiter plate. The positive control reference sera used on each ELISA plate derived from the day 42 serum sample from one of each of group 7 CoV-2 RBD in MAS-1 and Group 9 CoV-1 RBD in MAS-1 euthanized mice, and the negative control derived from day 42 MAS-1 placebo used in each ELISA plate showed a high degree of conformity between analyses with respect to the assigned titer for the positive reference and to the Mean baseline OD±3×SD “Threshold” for the MAS-1 placebo negative control across all analyses through day 168 (FIGS. 1A, 2,A, 2B, and 2C).

In Summary: The data in Examples 1 and 2 indicate that alum adsorbed CoV-2 RBD Group 3 sera showed a greater degree of variability between individual mice than either of the MAS-1 adjuvanted Groups 7 and 9. Also, at the same 10 μg CoV-2 RBD. MAS-1 adjuvanted Group 7 mean minimum log end-point titers, for the remaining 5 animals per group, remained close to 2 logs higher than those promoted by alum adjuvanted Group 3 titers with values on day 168 of 5.60 vs. 3.78, respectively, with a similar response observed with MAS-1 adjuvanted CoV-1 RBD. Thus. MAS-1 promotes a robust and durable humoral immune response towards either CoV-2 RBD or CoV-1 RBD with no diminution through day 168.

The data suggest that the MAS-1 adjuvanted vaccines of the present disclosure have potential to provide improved durability of the immune response towards Covid infection Significantly, currently approved SARS-CoV-2 vaccines demonstrate waning of titers within a few months, and require booster doses recommended by the CDC after 4 to 6 months after two doses of mRNA-based Covid-19 vaccines, and after as little as 2 months after a single dose of adenovirus-vectored Covid-19 vaccine (CDC, Jan. 4, 2022). More recently, a fourth dose of mRNA vaccine has been recommended in individuals 50 years and older to offset further waning of immunity in boosted individuals towards VOCs, particularly against the Omicron VOC (Watson. C (Feb. 3, 2022) Nature 602: pp 17-18 “Three, four or more boosters: what's the magic number for booster shots”; Levine-Tiefenbrum, M (February 2022) MedRxiv https://doi.org/10.1101/2021.12.27.21268424). Thus, there is a burning need for improved Covid-19 vaccines having a more durable response, in particular, a more durable response towards VOCs.

### Example 3: Potential for MAS-1 to Enhance Cross-Reactive Immunity Towards Emergent CoV-2 Variants of Concern (VOCs)

Covid-19 viral evolution presents significant challenges for ongoing vaccine development. Since the first emergence of the Covid-19 pandemic in Wuhan, the SARS CoV-2 virus has evolved to give rise to a number of so called variants of concern (VOC). The wild type WT-D614[G] strain, on which the first generation of anti-Covid-19 vaccines are based, and subsequent Alpha variant (strain B.1.1.7), were the dominant strains circulating in the US, UK, and elsewhere at the beginning of the Covid-19 pandemic in early 2020. The Beta VOC (strain B.1.351) became dominant in South Africa in the fall of 2020 and early in 2021. Both these strains were superseded by VOCs that emerged later in 2021, firstly by the Delta VOC (strain B.1.617.2), and subsequently by the Omicron VOC (strain B.1.1.529 BA.1 and more recently BA.2), a strain that also first emerged in South Africa and that is now the dominant VOC worldwide. Sera isolated from individuals infected with B.1.1.7 and B.1.351 variants or immunized with the Pfizer-BNT mRNA vaccine were reported to express significantly reduced titers towards the B.1.351 Beta variant compared with those towards the B.1.1.7 strain (Planas, D., Bruel, T., et al., Mar. 26, 2021 Nature Medicine 27: 917-924 “Sensitivity of infectious SARS-CoV-2 B.1.1.7 and B.1.351 variants to neutralizing antibodies”). Whereas the sera were comparably effective towards the D614[G] and B.1.1.7 variants, the neutralizing titers of convalescent sera towards the B.1.351 VOC were reduced at least 6-fold and those from individuals immunized with the Pfizer mRNA vaccine were reduced 14-fold. The Beta variant compared with the WT D614[G] strain has 5 notable mutations in the Receptor Binding Domain (RBD and 3 in the N-Terminal domain (NTD) of the Spike protein that are associated with antibody escape (B. L. Sievers et al., Jan. 13, 2022 Sci. Transl. Med. 10.1126/scitranslmed.abn7842). Whereas, the Delta (B.1.617.2) variant has a single important mutation (L452R) relative to the D614G WT virus that is associated with antibody escape, the Omicron B.1.1.529 variant has 11 mutations in the NTD and 15 in the RBD relative to the WT D614G strain. As a result, it was predicted that the Omicron variant would, like the Beta variant, exhibit antibody escape relative to the WT D614G strain. Sievers et al., report that convalescent sera from individuals infected with WT D614 were as reactive towards the Delta strain as towards the WT D614[G] strain, but significantly reduced towards both Beta and Omicron. Similarly, in subjects vaccinated with either Pfizer of Moderna mRNA vaccines, both being based on WT D614[G] Spike protein, antibody responses towards both Beta and Omicron variants were reduced compared with those towards the WT D614[G] and Delta variants. Nevertheless, although relative titers were reduced towards Beta and Omicron, the vaccines, especially after a third (and even 4^{th} booster) dose were effective at protecting from serious infection and death caused by Beta and Omicron variants. Nevertheless, as discussed by Sievers et al.,, (B. L. Sievers et al., Jan. 13, 2022 Sci. Transl. Med. 10.1126/scitranslmed.abn7842) and Watson (Watson. C (Feb. 3, 2022) Nature 602: pp 17-18) future protection requires that Covid vaccines will need to have both broader and more durable immune responses to combat future variants.

The Spike protein of the coronavirus binds with the ACE2 receptor to initiate infection of human cells by the virus. The RBD from the Spike proteins of the original SARS CoV-1 and the current SARS CoV-2 responsible for Covid-19 infections represent a comparatively extreme example of the range of potential structural differences between functional coronavirus Spike proteins. Thus, an assessment of the cross-reactivity of CoV-2 RBD-specific antisera to cross-react with the CoV-1 RBD, and vice versa, provides a measure to assess the potential of a particular Covid-19 vaccine to induce broad cross-reactive protection towards emergent CoV-2 VOCs.

The reactivity of the sera from Groups 3 and 7 was assessed by ELISA towards their vaccine matched CoV-2 RBD target antigen and for cross-reactivity towards the CoV-1 RBD heterologous antigen. Similarly, Group 9 sera was assessed against the matched CoV-1 RBD and for cross-reactivity towards the CoV-2 RBD.

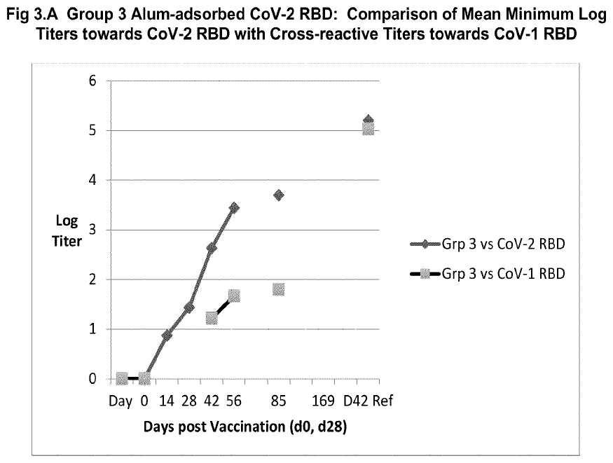

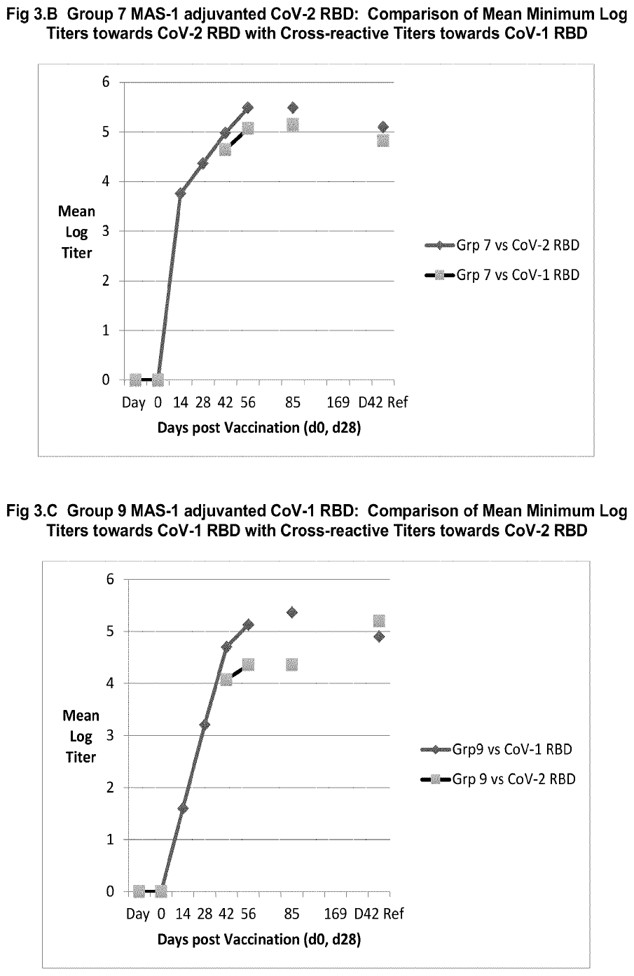

Mean minimum log end-point titers for Group 3 alum-adjuvanted CoV-2 RBD positive control sera against the matched CoV-2 RBD and for cross-reactivity towards CoV-1 RBD are presented in FIG. 3.A. Mean minimum log end-point titers for Group 7 MAS-1-adjuvanted CoV-2 RBD against matched CoV-2 RBD and for cross-reactivity towards CoV-1 RBD are presented in FIG. 3.B. Mean minimum log end-point titers for Group 9 MAS-1 adjuvanted CoV-1 RBD sera against matched CoV-1 RBD and for cross-reactivity towards CoV-2 RBD are presented in FIG. 3.C.

The mean minimum log end-point titers for Group 3 sera at days 42, 56, and 84 show that alum-adsorbed CoV-2 10 μg RBD induced cross-reactive titers that towards CoV-1 RBD were approximately two logs lower at approx. log 1.9 than those expressed towards the “vaccine-matched” CoV-2 RBD target antigen at approx. log 3.9 by day 85 (FIG. 3.A).

The mean minimum log end-point titers for Group 7 sera at days 42, 56 and 84 show that MAS-1 adjuvanted CoV-2 10 μg RBD induced highly cross-reactive titers towards CoV-1 RBD that at approx. log 5.1 were almost equivalent to those expressed towards the “vaccine-matched” CoV-2 RBD target antigen at approx. 5.5 by day 85 (FIG. 3.B).

The mean minimum log end-point titers for Group 9 sera at days 42, 56 and 84 show that MAS-1 adjuvanted CoV-1 10 μg RBD induced cross-reactive titers that towards CoV-2 RBD at approx. log 4.4 were approximately 1 log lower than those expressed towards the “vaccine-matched” CoV-1 RBD target antigen at approx. log 5.4 by day 85 (FIG. 3.C), but which were still higher than those induced by alum-adjuvanted CoV-2 RBD towards the matched CoV-2 RBD (FIG. 3A) at log 3.9 at day 85, indicating that MAS-1 is promoting a meaningful degree of cross-reactive immunity when formulated with both CoV-1 and CoV-2 RBD antigens and thereby exhibits significant promise for development of Covid vaccine protection against emergent VOCs.

Day 42 reference standard sera for Group 7 and Group 9 run on their respective microtiter plates were highly reproducible, providing confidence in the titer assessments shown in FIGS. 3A, 3B, and 3C. To the extent that MAS-1 adjuvanted CoV-2 and CoV-1 RBDs induce neutralizing titers towards live coronavirus, these cross-reactive ELISA data support that MAS-1 adjuvanted CoV-2 RBD would have potential to provide a robust and durable response towards emergent genetic variations of the SARS CoV-2, virus. These data further suggest that a MAS-1 adjuvanted vaccine comprising a combination of both CoV-1 and CoV-2 RBDs (or their trimeric Spike proteins) may also have potential to provide robust broad spectrum and durable protective immunity.

In Summary: MAS-1 induces an antibody response with both CoV-2 and CoV-1 RBDs that exhibits robust broad-based cross-reactivity towards the respective counterpart CoV RBDs. To the extent that MAS-1 adjuvanted CoV-2 and CoV-1 RBDs induce neutralizing titers towards live coronavirus, these cross-reactivity data support that MAS-1 adjuvanted CoV-2 RBD shows significant potential to provide a robust and durable response towards emergent genetic variations of the virus occurring as a result of mutation to reduce the potential for immune evasion thereby prolonging the useful lifetime of the vaccine.

### Example 4: Assessment of MAS-1-Induced In Vitro Pseudovirus Neutralization Activity and Cross-Neutralization Activity Towards Covid-19 VOCs

A virus neutralization assay (VNA) was performed at the Baylor College of Medicine. School of Tropical Medicine, to assess the pooled sera from Groups 3, 7, 9 and a 1:1 combination of groups 7+9 at days 42 and 84 for their potential in vitro to neutralize the ability of the virus to infect human cells.

Procedure: To this end, serial dilutions of each MAS-1 adjuvanted test Group's antisera were assayed in vitro for their ability to neutralize the infection of human cells by pseudo viruses carrying the Spike proteins of the original WT-D614G (wild type CoV-2 virus) and the Beta VOC (CoV-2 strain B.1.351) first reported in South Africa to assess the potential for MAS-1 adjuvant to enhance durable and cross-reactive antibody responses towards VOCs.

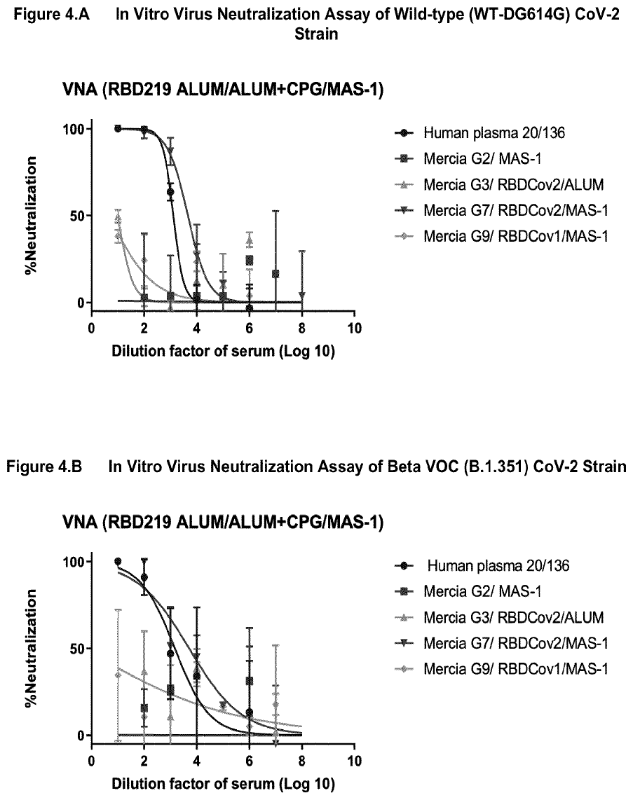

Results: Data presented in FIG. 4.A show the neutralization titers towards the WT-D6140, strain, the dominant strain circulating in the US, UK and elsewhere at the beginning of 2020 Covid-19 pandemic. Data presented in FIG. 4.B shows the neutralization titers towards the Beta (B.1.351) VOC strain that became dominant in South Africa in the fall of 2020. Titers for convalescent sera are included as positive control in both assays. The neutralizing titers are summarized below:

Neutralizing Titers Vs. CoV-2 WT-D614G Strain:
Convalescent plasma (control)=3.69E+03
Group 3 CoV-2 RBD-alum (positive control)=1.0E+01
Group 7 CoV-2 RBD in MAS-1=4.62E+03
Group 9 CoV-1 RBD in MAS-1=<1.0E+01
   Neutralizing Titers Vs. CoV-2 SA B.1.351 Strain:
Convalescent plasma (control)=1.47 E+03
Group 3 CoV-2 RBD-alum (positive control)=<1.0E+01
Group 7 CoV-2 RBD in MAS-1=5.13E+03
Group 9 CoV-1 RBD in MAS-1=<1.0E+01

The CoV-2 RBD used in the current study is derived from the original WT D614G strain as are the Spike and RBDs used in all currently approved Covid vaccines. The CoV-2 RBD in MAS-1 (Group 7) sera showed excellent neutralizing activity against both the WT D614G and Beta (SA B.1.351) strains compared with convalescent sera. The Beta variant (strain B.1.351), is reported by Sievers et al (B. L. Sievers et al., Jan. 13, 2022 Sci. Transl. Med. 10.1126/scitranslmed.abn7842) to be significantly less responsive to antibodies raised by current approved Covid-19 vaccines that are all based on the WT D614[G] Spike protein or its RBD. Consistent with the observations reported by Sievers et al., the susceptibility of the Beta B.1.351 VOC to the convalescent control sera shown above was significantly reduced compared to that towards the WT D614G strain at 3.69E+03 vs 1.47 E+03, respectively. By contrast, the Beta VOC was equally susceptible to the Group 7 MAS-1 adjuvanted CoV-2 RBD sera as was the WT D614G strain with neutralizing titers of 5.13E+03 vs 4.62E+03, respectively. These in vitro cross-neutralization assay data presented in FIG. 4A and FIG. 4B towards the WT DG614G and Beta B.1.351 VOC are consistent with the highly cross-reactive ELISA data exhibited by Group 7 MAS-1 adjuvanted CoV-2 RBD sera towards the heterologous CoV-1 RBD which indicated the potential for MAS-1 induced Covid vaccines to exhibit a high degree of cross-reactivity with Covid VOCs.

### Cross Reactivity and Durability of the Neutralizing IgG Response

Table 1 below shows the log IC50 neutralizing antibody titer values of SARS CoV-2RBD/MAS-1 and convalescent plasma induced by the original Wuhan strain of vaccinated mice on day 168 after prime vaccination for Wuhan. Omicron. Delta, Beta and SARS CoV-1:

TABLE 1
Neutralizing Antibody Levels (IC50 values) of mouse
sera collected day 168 after prime vaccination

| | | Convalescent | |
|---|---|---|---|
| | Cntrl Bkgd | plasma | MAS-1 |
| VOC strain | Day 168 Log IC50 Neutralizing IgG | | |
| Wuhan | 0.0 | 4.0 | 4.0 |
| Omicron | 0.0 | 0.8 | 0.0 |
| Delta | 0.0 | 3.1 | 3.0 |
| Beta | 0.0 | 2.7 | 3.3 |
| SARS CoV-1 | 0.0 | 1.1 | 0.5 |

These data show that MAS-1 induces potent, durable, cross-reactive IgG response towards CoV-2 RBD antigen in BALB/c mice, with no diminution of neutralizing titers through 6 months post-vaccination. Additionally, the neutralizing titers cross-reactive with variants, including Beta VOC, were comparable or superior to human convalescent sera induced by the original Wuhan strain.

In Summary: The data in Examples 3 and 4 indicate that MAS-1 induces a robust, durable and more broadly protective immune response than that provided by natural exposure (convalescent sera) or as reported by Sievers et al., or the current generation of approved Covid-19 vaccines based on the WT D614 strain. These characteristics suggest that MAS-1 has potential to provide broad-based durable protection towards existing VOCs and, therefore, possibly towards VOCs yet to emerge for purposes of pandemic planning when combined with the appropriate Covid-19 RBD or Spike antigen.

Although it is difficult to predict the outcomes of combining any particular antigen with any particular adjuvant, MAS-1 adjuvanted seasonal inactivated influenza virus (IIV) antigens did improve the robustness and durability of immune protection, including towards both vaccine and non-vaccine viral strains, in clinical studies in both the general adult population (Phase 1A; 18-49 years) and at-risk elderly subjects (Phase 1B/1B extension; 65 years and older), a population also particularly at-risk for serious Covid-19 infection. See U.S. Patent Application Publication No. 2012/0219605, incorporated herein by reference in its entirety; and Gorse et al., 2022 Vaccine 40: 1271-1281 (A phase 1 dose-sparing, randomized clinical trial of seasonal trivalent inactivated influenza vaccine combined with MAS-1, a novel water-in-oil adjuvant/delivery system) and Gorse eta al., 2022 Vaccine 40; 1472-1482 (MAS-1, a novel water-in-oil adjuvant/delivery system, with reduced seasonal influenza vaccine hemagglutination dose may enhance potency, durability and cross-reactivity of antibody responses in the elderly). Thus, the data with CoV-2 and CoV-1 RBDs in MAS-1 described herein suggest that MAS-1 has the potential to improve the durability and cross-reactivity of protection from Covid-19 in human subjects, including elderly subjects who are among the most vulnerable to serious infections form both Covid-19 and influenza viral infections.

### Example 5: Th1/Th2 Specificity of the Immune Response by IgG Isotype Analyses and Potential Consequences for Covid-19 Infection

Background: Although associated with various other infections, including influenza among others, Covid-19 serious infections, often leading to death, especially in vulnerable at-risk individuals, including the elderly, infected by SARS CoV-2 have illustrated the critical role for an effective host immune response and the serious consequences of host immune dysregulation and the so-called “cytokine storm” for both short-term and long-term effects. (Tan et al., Frontiers Immunol. (30 September 2021) “Hyperinflammatory Immune Response and Covid-19: A Double Edged Sword). Early activation of type 1 interferon (IFN) mediated innate immunity following infection is a key event for protection, and viral interference with this process can enhance viral replication and lead to host hyperinflammation and cytokine storm (Tan 18). Subsequent adaptive immunity including activation of antibody producing B cells, CD4+ and CD8+ T cells are each primed to control pathogenic infection. Covid-19 severity has been associated with a Th1/Th17 biased cytokine storm, including IL-1Beta, IL-2R, IL-6, IL-17, and TNF-Alpha, associated with dysregulation of the immune response and disease severity (Tan 43, 45). To what extent Long-Covid (or Long Haul) symptoms can be attributed to dysfunction of the host immune response to Covid-19 infection is not clear, but the broad range of symptoms associated with Long Haul Covid suggest a common link mediated via the host's immune response resembling an autoimmune condition is likely involved, as well as perhaps contributing to impairment of the blood brain barrier and microglial activation leading to neurological symptoms (Tan 81, 95, 96). Various attempts have been employed to block the effects of these cytokines in attempts to mitigate the effects of the hyperinflammation and associated cytokine storm, but there is a limited window of opportunity at the outset of hyperinflammation to effect these interventions. In addition, it has been theorized that hyper immunity, ad particularly CD8 hyper inflammation and hyper inflammatory microglia might be involved in triggering Long Covid. See Mesa, N., “Multiple Possible Causes of Long COVID Come into Focus;” The Scientist, Oct. 10, 2022. These observations suggest that a Covid-19 vaccine that promotes a more balanced immune response may prime the host immune response to respond to infection with a more balanced humoral and cellular response, thereby mitigating the risks for developing host-mediated hyperinflammation and subsequent sequelae. To the extent that the host inflammatory response is associated with Long Covid, it is possible that vaccination priming a balanced immune response to reduce the risks for Th1/Th17 mediated hyperinflammation might reduce the risk for developing Long Haul Covid following “break-through” infection and, perhaps, might even have an immunotherapeutic benefit in subjects already exhibiting symptoms of Long Haul Covid. The effects of currently approved vaccines on the incidence of Long Haul Covid are reportedly modest at best or none at all (Ledford, H “How Vaccination Effects the Risk of Long Covid” Nov. 23, 2021 Nature 599 pp 546-547).

Adjuvants are frequently used to promote the immune response to antigens which by themselves are poorly immunogenic. Alum adjuvant, the most clinically used adjuvant, primarily generates a Th2 response. The oil-in-water adjuvants MF59 and AS03 are both approved for use in influenza vaccines, Fluad (El Sahly H. MF59™ as a vaccine adjuvant: a review of safety and immunogenicity. Expert Rev Vaccines 2010; 9:1135-41. Doi.org/10.1586/erv.10.111) and Pandemrix (McElhaney J E, Beran J, Devaster J-M, Esen M. Launay O. Leroux-Roels G, et al. AS03-adjuvanted versus non-adjuvanted inactivated trivalent influenza vaccine against seasonal influenza in elderly people: a phase 3 randomised trial. Lancet Infect Dis 2013; 13:485-96), respectively, induce proinflammatory cytokines and chemokines (Wilkins, A L et al. Frontiers in Immunol Dec. 13, 2017 1 AS03- and MF59-Adjuvanted Influenza Vaccines in Children). Other adjuvants such as Toll-like receptor (TLR) agonists, including CpG, can shift the immune balance by, for example, stimulation of type 1 interferon (IFN) expression to promote Th1 responses. Type 1 IFN responses are induced by mRNA vaccines and thereby promote a Th1 biased response (Cagigi, A and Lore, K Jan. 18, 2021 Vaccines 9: 61-75 Immune Responses Induced by mRNA Vaccination in Mice, Monkeys and Humans).

The immune response promoted by MAS-1 with CoV-2 RBD and Spike protein was compared herein with those promoted by alum adjuvanted CoV-2 RBD as control.

### Results: Specificity of the Immune Response by IgG Isotype Analyses

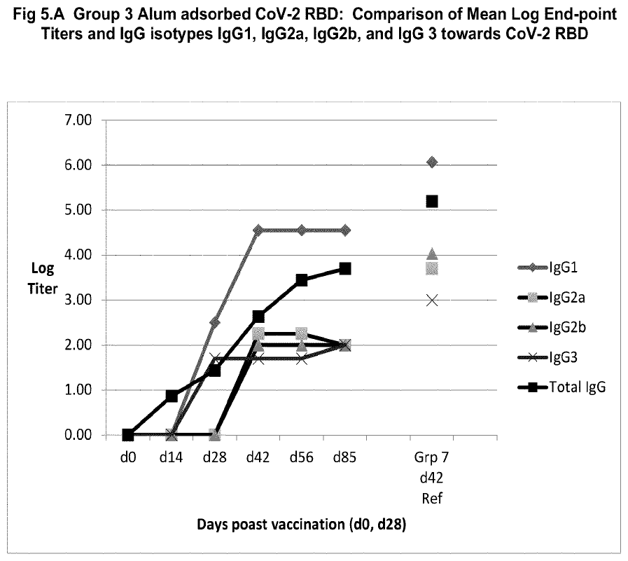

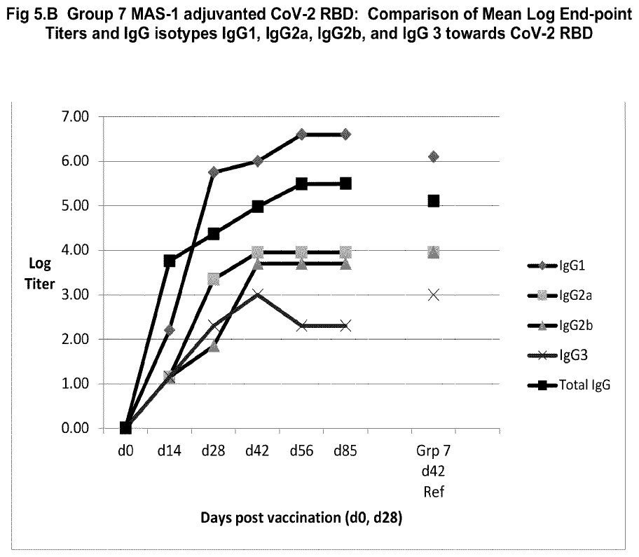

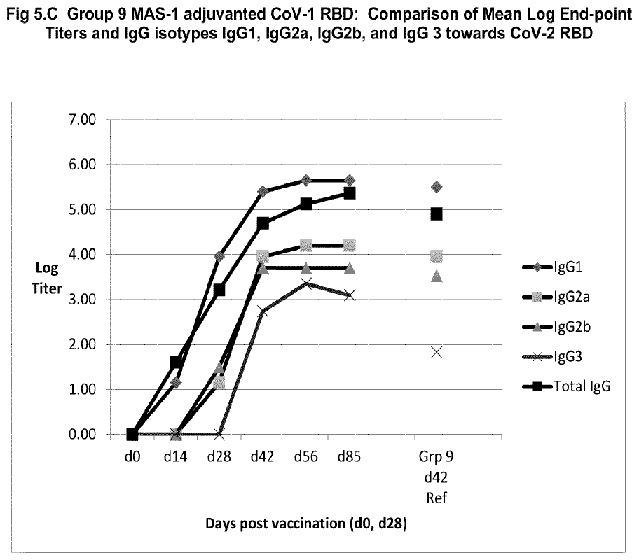

The IgG isotype response (Th2 type IgG1 and IgG2b; Th1 type IgG2a and IgG3) for groups 3, 7, and 9 was determined at days 14, 28 (pre-dose 2), 42, 56, and 84 to assess the Th2/Th1 balance of the humoral immune response. IgG isotype analyses were performed on pooled sera from groups 3, 7 and 9 through day 85. IgG isotype log minimum end-point titers for group 3 sera against CoV-2 RBD are presented in FIG. 5.A. Alum adsorbed CoV-2 RBD induces a Th2 dominant IgG response with IgG1 achieving log titer of 4.5 by day 42 (+14 days post dose 2). IgG2a, IgG2b and IgG3 titers were comparable and achieved a log titer of approximately 2.0 by day 42. All isotype titers remained constant through day 85 thus far, whereas, total mean log end-point titers were seen to continue to rise though day 85 achieving a mean log titer of 3.7. The Th2/Th1 balance based on relative IgG1/IgG2a titers on day 85 was 4.5:2.0=2.27.

IgG isotype minimum mean log end-point titers for Group 7 sera against CoV-2 RBD are presented in FIG. 5.B. MAS-1 adjuvanted CoV-2 RBD induces a Th2 biased IgG response with IgG1 achieving log titer of approximately 6.0 by day 42 (+14 days post dose 2), rising to 6.6 by day 85. IgG2a and IgG2b achieved log titers of approximately 4.0 by day 42 and remained constant through day 85. IgG3 titers were slightly lower and achieved a log titer of between 2.3 and 3.0 between days 42 and 85. All isotype titers remained constant through day 85 thus far, whereas, total IgG titers peaked by day 56 and remained constant thereafter achieving a mean log titer of 5.49 by day 85. The Th2/Th1 balance based on relative IgG1/IgG2a titers on day 85 was 6.0:3.95=1.67, indicating a more balanced Th2/Th1 response than that observed in response to Group 3 alum-adsorbed CoV-2 RBD control.

IgG isotype minimum mean log end-point titers for Group 9 sera against CoV-1 RBD are presented in FIG. 5.C. MAS-1 adjuvanted CoV-1 RBD induces a Th2 biased IgG response with IgG1 achieving log titer of approximately 5.40 by day 42 (+14 days post dose 2), rising to 5.65 by day 85. IgG2a and IgG2b achieved log titers of approximately 4.0 by day 42 and remained constant through day 85. IgG3 titers were slightly lower and achieved a log titer of between 2.74 and 3.35 between days 42 and 85. All isotype titers remained constant through day 85 thus far, whereas, total IgG titers were seen to continue to rise though day 85 achieving a mean log titer of 5.50 by day 85. The Th2/Th1 balance based on IgG1/IgG2a on day 85 was 5.65:4.20=1.35.

In summary: IgG isotype analyses showed that alum adsorbed CoV-2 RBD induced a Th2 dominant response with Th2/Th1 log ratio of 2.27, whereas, MAS-1 adjuvanted CoV-2 and CoV-1 RBDs induced more balanced isotype profiles with Th2/Th1 log ratios of 1.67 and 1.35, respectivey. MAS-1 adjuvated CoV-2 RBD induced Th1 IgG2a minimum log end-point titers of 3.94 by day 42 that remained stable through day 85, and exceeded the total log liters induced by alum adsorbed CoV-2 RBD from days 42 through day 85 which rose from 2.63 to 3.70, respectively. Similarly, MAS-1 adjuvanted CoV-1 RBD induced Th1 IgG2a log titers of 3.95 and 4.20 between days 42 and 85, comparable to the Th1 IgG2a log titers induced by MAS-1 adjuvnated CoV-2 RBD at 3.95.

### Example 6 Comparative Adjuvant Study: Comparison of MAS-1 Adjuvanted CoV-2 RBD (3 μg Dose) and Spike (1 μg Dose) with AS03 and MF59 Adjuvants

In a confirmatory follow up study. MAS-1 adjuvant with a) CoV-2 RBD antigens (original Wuhan strain) and b) full length trimeric Spike (“Spike”) antigens (original Wuhan strain) were compared to ASO3-like and MF59 (Addavax) in a collaborative study undertaken in a different strain (C57BJ/6J) of mice as part of an NIH sponsored adjuvant comparator evaluation performed at the Univ of Montana. End-point titers determined towards target RBD were assessed for the various treatment groups. Evaluations of the specificity of the response were evaluated by IgG isotype analyses for Th2 IgG1 and Th1 IgG2c titers. The cellular response stimulated by the adjuvants was evaluated by determining the cytokine profile expressed by isolated splenocytes and by adjacent lymph nodes.

Procedure: Mice were given two injections (100 μl injection i.m., 50 μl in each rear calf muscle) containing 3 μg RBD or 1 μg Spike 21 days apart. ELISA assays for RBD- or Spike-specific serum antibodies were performed at 21 days after the 2^{nd} injection. Spleen and draining LN cells were collected 21 days after 2^{nd} injection and cultured with RBD or Spike for 72 hours. Cultured supernatants were tested for cytokines using a multiplex ELISA kit (MesoScale Discovery).

Results: The Humoral Response Profile results are shown in Table 2 below:

TABLE 2
Humoral Response Profile

| | | | | MAS-1 | | |
|---|---|---|---|---|---|---|
| | | Cntrl | Ag*** | Log | AS03- | MF59** |
| | | Bkgd | alone | Titer | like* | (Addavax) |
| RBD | Total | 2.0 | 3.7 | 7.5 | 4.5 | 4.5 |
| | IgG | | | | | |
| | IgG2c | 2.0 | 1.7 | 6.4 | 2.0 | 3.0 |
| | (Th1) | | | | | |
| | IgG1 | 1.5 | 3.9 | 6.5 | 4.8 | 5.0 |
| | (Th2) | | | | | |
| Spike | Total | 1.5 | 5.0 | 6.5 | 5.8 | 5.9 |
| | IgG | | | | | |
| | IgG2c | 1.5 | 3.0 | 6.0 | 2.5 | 4.5 |
| | (Th1) | | | | | |
| | IgG1 | 1.5 | 5.3 | 7.2 | 6.2 | 6.4 |
| | (Th2) | | | | | |

*The AS03 adjuvant employed in this study was not GMP grade. It is identical to AS03 in composition but was not necessarily manufactured in accordance with GMP standards.
**The MF59 employed in this study was the animal-use version known as Addavax.
***Non-adjuvanted Antigen alone Control.

The results showed that MAS-1 induced potent IgG titers towards CoV-2 RBD and Spike antigens that were superior to either AS03 or MF59 oil-in-water adjuvants in C57BL/6J mice. This is particularly more evident with RBD than with the inherently more immunogenic Spike antigen.

MAS-1 adjuvanted CoV-2 RBD produced the highest level of anti-RBD IgG and anti-Spike, outperforming both ASO3 and MF59 (Addavax). As can be seen in Table 2, unadjuvanted RBD and Spike induced Th2 biased IgG responses, and both AS03 and MF59 promoted a more Th2 biased IgG response. In contrast, the data confirmed that based on isotype profiles, MAS-1 induced a relatively balanced Th2-type IgG1 to Th1-type IgG2c humoral response compared to either AS03 or MF59 which were dominated by the Th2-type IgG 1 response.

The Cellular Response Profile results are shown in Table 3 below:

TABLE 3
Cellular Response Profile

| | | Cntrl | Ag | MAS-1 | AS03- | MF59 |
|---|---|---|---|---|---|---|
| | | Bkgd | alone | pg/mL | like | (Addavax) |
| IFNg | Draining | 0 | 0 | 3000 | 2000 | 1000 |
| | LN | | | | | |
| | Spleen | 0 | 0 | 300 | 25 | 75 |
| IL-17A | Draining | 0 | 250 | 200 | 300 | 200 |
| | LN | | | | | |
| | Spleen | 0 | 0 | 3 | 2 | 17 |
| IL-5 | Draining | 0 | 50 | 200 | 1800 | 1500 |
| | LN | | | | | |
| | Spleen | 0 | 100 | 100 | 1000 | 1000 |
| TNFa | Draining | 200 | 100 | 500 | 300 | 275 |
| | LN | | | | | |
| | Spleen | 150 | 75 | 150 | 120 | 140 |
| IL-10 | Draining | 50 | 75 | 200 | 500 | 400 |
| | LN | | | | | |
| | Spleen | 100 | 100 | 300 | 350 | 350 |
| IL-2 | Draining | 50 | 80 | 40 | 80 | 100 |
| | LN | | | | | |
| | Spleen | 50 | 40 | 110 | 110 | 180 |

The data evaluated the cellular immune response based on the expression of the cytokines IFNγ IL-5 IL-10, IL-17, TNF-a IL-2. These data show that MAS-1 induces a Th1 type cellular response, inducing more IFNg than AS03 or MF59 with RBD and Spike. In addition, MAS-1 did not stimulate IL-17, which is associated with the hyperinflammatory cytokine storm. MAS-1 also induced a modest IL-10 response, and MAS-1 did not induce IL-5, whereas both AS03 and MF59 induced IL-5 which is associated with eosinophil activation.

The cytokine profiles showed that MAS-1 enhances IFNγ-producing cells, indicating the development of Th1 cells, and that MAS-1 does not to induce Th17 cell development associated with the Th1/Th17 hyperinflammatory response and cytokine storm. MAS-1 adjuvanted RBD did stimulate a modest increase in IL-10 which along with expression of IFNγ is consistent with stimulation of a balanced immune rather than a response dominated by pro-inflammatory Th1 or Th17 cytokines

In Summary: These data show a clear IgG isotype and cytokine analysis that corroborates the data presented in Examples 1, 2, and 5 above performed in BALB/cJ mice, confirming that MAS-1 promotes a balanced Th1/Th2 immune response that should be both preventive of infection, and protective against host immunopathology, and further supports the possibility that a MAS-1 adjuvanted Covid vaccine may have potential for therapeutic intervention in Covid, and Long Haul Covid.

In addition, MAS-1 demonstrates significant dose sparing, enhanced durability and cross-reactive protection towards Covid viral strains, and MAS-1 can suppress hyperinflammatory responses by restoring balance to the immune system demonstrating immunotherapeutic potential for certain autoimmune and inflammatory conditions (e.g. Long Haul Covid).

### Example 7: Point of Use Formulation

Preparation of the MAS-1 adjuvanted vaccine at point-of-use (POU), rather than in bulk, allows for a versatile formulation of vaccine with any candidate SARS-CoV-2 viral antigen without requiring large scale commitment of precious candidate antigens to formulations which may not result in “protective” immunity. A single vial of sterile MAS-1 adjuvant vehicle is typically combined with 0.5 ml of sterile aqueous antigen solution to produce 1.9 mL vaccine emulsion. The dose volume will be a maximum of 0.3 mL dose in elderly subjects, and 0.2 mL or possibly 0.1 mL in younger adults, thereby providing at least 4 doses per vial of 0.3 ml dose, at least 6 to 7 doses per vial of 0.2 mL dose, or 10 to 12 repeat doses at 0.1 mL dose volumes. Based on formulation validation studies on file with the FDA, although the physic-chemical integrity of the emulsions remains stable for a minimum of 48 hours at room temperature, in order to minimize the risk for microbial contamination, the doses derived from a single vial of the vaccine emulsion prepared POU are administered within 4 hours post-emulsification.

While various embodiments of the present invention have been shown and described herein, it is emphasized that such embodiments are provided by way of example only. Numerous variations, changes and substitutions may be made without departing from the invention herein in its various embodiments. Specifically, when any range is described herein, unless clearly stated otherwise, that range includes all values therein and all sub-ranges therein.

Also, and more generally, in accordance with disclosures, discussions, examples and embodiments herein, there may be employed conventional molecular biology, cellular biology, microbiology, and recombinant DNA techniques within the skill of the art. Resources incorporated by reference herein are for their respective content and teachings found therein. Such incorporation, at a minimum, is for the specific teaching and/or other purpose that may be noted when citing the reference herein. If a specific teaching and/or other purpose is not so noted, then the published resource is specifically incorporated for the teaching(s) indicated by one or more of the title, abstract, and/or summary of the reference. If no such specifically identified teaching and/or other purpose may be so relevant, then the published resource is incorporated in order to more fully describe the state of the art to which the present invention pertains, and/or to provide such teachings as are generally known to those skilled in the art, as may be applicable. However, it is specifically stated that a citation of a published resource herein shall not be construed as an admission that such is prior art to the present invention. Also, in the event that one or more of the incorporated published resources differs from or contradicts this application, including but not limited to defined terms, term usage, described techniques, or the like, this application controls as a preferred embodiment, and any contradiction may be viewed as an alternative embodiment.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

1. A water-in-oil nanoemulsion vaccine composition comprising an aqueous phase and an oil phase, wherein:
the aqueous phase comprises:
25% to 35% by weight of the emulsion, and is in the form of aqueous nanoglobules having a median diameter from about 0.3 μm to about 1 μm; and
one or more SARS-CoV-2 antigens; and
the oil phase comprises:
from 65% to 75% by weight of the emulsion, which oil phase comprises 85% to 90% of squalene and squalane, 9% to 12% mannide monooleate, and 0.5% to 0.7% polyoxyl-40-hydrogenated castor oil, each by weight of the oil phase.

2. The nanoemulsion vaccine composition of claim 1, wherein the SARS-CoV-2 antigen is a protein antigen.

3. The nanoemulsion vaccine composition of claim 1, wherein the composition comprises more than one SARS-CoV-2 antigen.

4. The nanoemulsion vaccine composition of claim 1, wherein the SARS-CoV2 antigen comprises components derived from more than one SARS-CoV-2 strain

5. The nanoemulsion vaccine composition of claim 1, wherein the SARS-CoV2 antigen comprises SARS-CoV-2 antigens derived from more than one SARS-CoV-2 strain.

6. The nanoemulsion vaccine composition of claim 1, wherein squalene comprises a range from about 40% to about 60% by weight of the oil phase.

7. The nanoemulsion vaccine composition of claim 1, wherein squalane comprises a range from about 40% to about 60% by weight of the oil phase.

8. The nanoemulsion vaccine composition of claim 1, wherein the squalene and squalane are in a ratio of about 1:1 by weight.

9. The nanoemulsion vaccine composition of claim 1, wherein the median globule size is about 300 nm.

10. The nanoemulsion vaccine composition of claim 1, wherein the composition has a viscosity of 100 cP or less.

11. The nanoemulsion vaccine composition of claim 1, wherein the aqueous phase further comprises a protein solubilizer.

12. The nanoemulsion vaccine composition of any claim 11, wherein the protein solubilizer is urea or DMSO.

13. A method for SARS-CoV-2 prophylaxis comprising administering to a patient in need thereof a vaccine composition according to claim 1.

14. A method for attenuating SARS-CoV-2 infection comprising administering to a patient in need thereof a vaccine composition according to claim 1.

15. A method for producing an immune response protective against SARS-CoV-2 infection comprising administering to a patient in need thereof a vaccine composition according to claim 1.

16. A method for long-haul SARS-CoV-2 prophylaxis comprising administering to a patient in need thereof a vaccine composition according to claim 1.

17. A method for attenuating one or more symptoms of long-haul SARS-CoV-2 infection comprising administering to a patient in need thereof a vaccine composition according to claim 1.

18. A method for producing an immune response protective against long-haul SARS-CoV-2 infection comprising administering to a patient in need thereof a vaccine composition according to claim 1.

19. A method for increasing the potency and/or durability of an immune response to a SARS-CoV-2 antigen, comprising administering the antigen in a water-in-oil nanoemulsion vaccine composition comprising an aqueous phase and an oil phase, wherein:
the aqueous phase comprises:
25% to 35% by weight of the emulsion, and is in the form of aqueous nanoglobules having a median diameter from about 0.3 μm to about 1 μm; and
the SARS-CoV-2 antigen; and
the oil phase comprises:
from 65% to 75% by weight of the emulsion, which oil phase comprises 85% to 90% of squalene and squalane, 9% to 12% mannide monooleate, and 0.5% to 0.7% polyoxyl-40-hydrogenated castor oil, each by weight of the oil phase.

20. A kit for the point-of-use administration of a water-in-oil nanoemulsion vaccine against a SARS-CoV-2 infectious agent comprising: a vial of an adjuvant oil comprising mannide monooleate, squalene and squalane, a vial of aqueous PBS comprising SARS-CoV-2 antigen, at least one syringe, a lipid resistant three-way stopcock, at least one needle, and a vial for storing the formulated water-in-oil nanoemulsion vaccine.

21. The kit of claim 20, wherein the kit is for the point-of-use administration of a nanoparticulate water-in-oil emulsion vaccine against said SARS-CoV-2 infectious agent comprising: a vial of an adjuvant oil comprising mannide monooleate, squalene and squalane, a vial of aqueous PBS for combining with an antigen, two syringes, a lipid resistant three-way stopcock, two needles and a vial for storing the formulated water-in-oil emulsion vaccine.

22. A kit for the point-of-use administration of a water-in-oil nanoemulsion vaccine against a SARS-CoV-2 infectious agent comprising: a vial of an adjuvant oil comprising mannide monooleate, squalene and squalane, a vial of aqueous PBS comprising SARS-CoV-2 antigen, at least one syringe, a lipid resistant three-way stopcock, at least one needle, and a vial for storing the formulated water-in-oil nanoemulsion vaccine; wherein the nanoparticulate water-in-oil emulsion vaccine produced by the combination of the kit components is a composition of claim 1.

23. A water-in-oil nanoemulsion vaccine composition produced by the components of the kit of any of claim 1.
